(19) United States
(12) Patent Application Publication
(10) Pub. No.: US 2014/0087363 A1
(43) Pub. Date: Mar. 27, 2014

(54) METHOD FOR GENERATING, STORING, TRANSPORTING, ELUTING AND DETECTING CLINICAL RELEVANT INFORMATION IN PLASMA USING FILTER PAPER

(71) Applicant: Morten Ruhwald, Copenhagen V (DK)

(72) Inventor: Morten Ruhwald, Copenhagen V (DK)

(73) Assignee: HVIDOVRE HOSPITAL, Hvidovre (DK)

(21) Appl. No.: 13/992,643

(22) PCT Filed: Dec. 9, 2011
(86) PCT No.: PCT/DK2011/050474

§ 371 Date: Dec. 6, 2013

Related U.S. Application Data
(60) Provisional application No. 61/421,549, filed on Dec. 9, 2010.

(30) Foreign Application Priority Data
Dec. 9, 2010 (EP) 10194430.4

(51) Int. Cl. G01N 33/569

(52) U.S. Cl. CPC G01N 33/5695
USPC 435/5; 435/7.92; 435/6.12

(57) ABSTRACT

The present invention relates to a method that enables simpler, easier and more accurate determination cell mediated immune (CMI) responses using the biomarker IP-10 together with a simple and safe “dried blood spot” filter paper method of storing and shipping samples. The method is useful for the diagnosis and prognostication of diseases and conditions that can be diagnosed and prognosticated by measuring correlates of IP-10.

### FIELD OF INVENTION

The disclosure described herein is a novel method for storing and transporting blood and plasma using filter paper. The method is useful for the diagnosis and prognostication of diseases and conditions that can be diagnosed and prognosticated by measuring correlates hereof expressed as biomarkers in biological fluids such as blood and plasma.

### BACKGROUND OF THE INVENTION

The concept that capillary blood, obtained from pricking the heel or finger and blotted onto filter paper, could be used to screen for metabolic diseases in large populations of neonates was introduced in Scotland by Robert Guthrie in 1963.

There are several fully automated high throughput systems available e.g. the Wallac system (Perkin Elmer, USA) and several types of filter paper have been optimized for this end use e.g. the FDA approved Whatman 903 (Whatman, USA).

In Mei et al. “Innovative Non- or Minimally-Invasive Technologies for Monitoring Health and Nutritional Status in Mothers and Young Children” J. Nutr. 131: 1631s-1636s, 2001 it is postulated that “any analyte or biomarker that can be measured from whole blood, serum or plasma also can be measured from dried blood on filter paper”.

WO2008064684 discloses a diagnostic test comprising mixing blood with a test compound that can interact with constituents of the blood and spotting said mixture on filter paper and subsequently analyzing said blood sample for e.g. biomarkers. The inventors of WO2008064684 measured a bundle of potential biomarkers, but surprisingly failed to verify the diagnostic potential of well-known blood/plasma biomarkers (e.g. IFN-γ and IL-2), when transferring these to the filter paper.

The currently commercially available diagnostic assays for infection with M. Tuberculosis (MTB) rely on determining the level of IFN-γ or other pro-inflammatory markers that are known to be very unstable at room temperature in plasma e.g. determined using ELISA.

Similarly, the currently available monitoring assays for infection with cytomegalovirus (CMV) also rely on determining the level of IFN-γ or other pro-inflammatory markers that are known to be very unstable in plasma with prolonged storage at ambient temperature.

The problem with plasma or similar samples is that they need to be refrigerated or frozen to avoid degrading of the diagnostic biomarker. Therefore it is a logistical challenge and economic burden to transport samples before analysis, and therefore most assays are done within driving distance from the patient i.e. centralization is impossible and throughout the world there are thousands of lab technicians who each week run small batches of ELISAs. This is inefficient, expensive and could lead to misdiagnosis of patients in labs where to few tests are done to uphold routine.

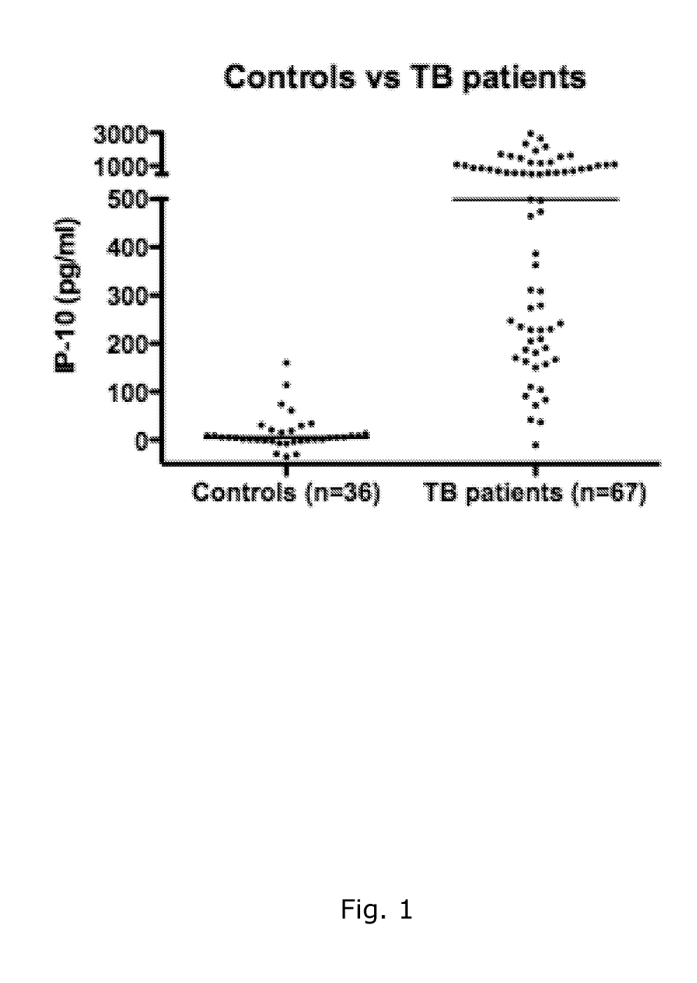

IFN-γ is the best-known biomarker for cell mediated immunity. PPD is a strong inducer of IFN-γ in CMI assays. The first version of the commercially available Quantiferon test was based on incubation of whole blood with PPD. One scientific paper of numerous examples of PPD as inducer of IFN-γ is [Brock I et al AJRCCM; 170:65-9, 2004]. As shown in FIG. 1 (lower panel) one can see the magnitude of IFN-γ response produced with PPD stimulation. This clearly demonstrates the clinical power of IFN-γ on plasma level.

However, in WO 2008/064684 (example 6) responses to PPD stimulation is detected in dried blood spots with e.g. IL-8 and MIP-1b, but the experiment fails to demonstrate that IFN-γ, which—as expected from the literature—should be induced in a responsive individual. On the contrary when compared to the control sample IFN-γ wobbles seemingly random up and down.

Another well-known biomarker for CMI is IL-2, e.g. induced by PPD (Sanchez et al Infection and immunity 1994), but as disclosed also in example 6 of WO 2008/064684, the inventors fails to demonstrate such an IL-2 response in whole blood on dried blood spots.

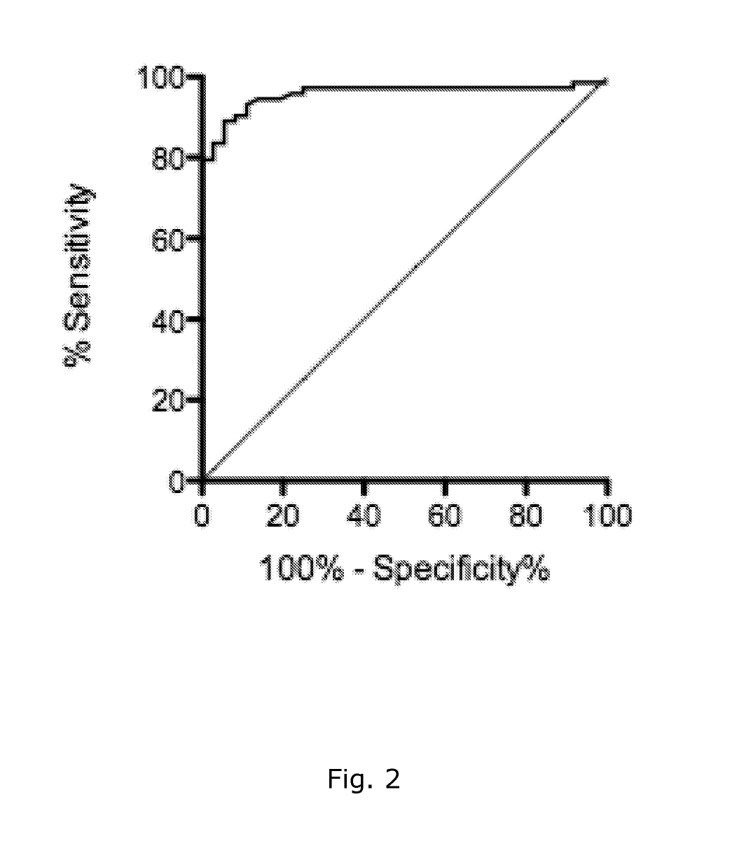

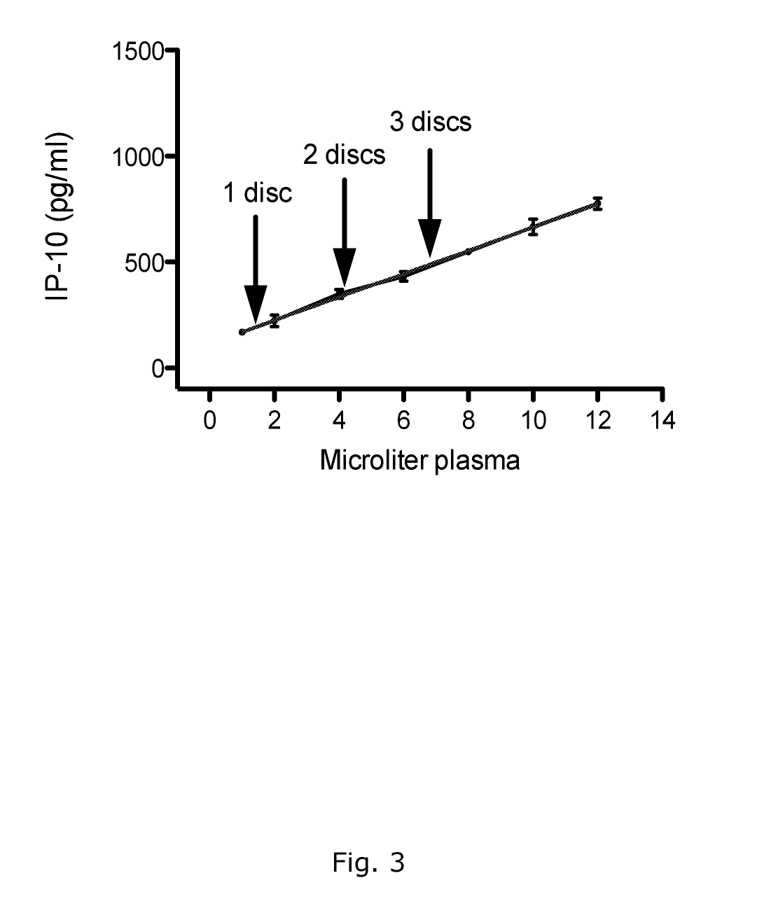

Another known biomarker for CMI is the highly expressed biomarker CCL2 aka MCP-1. E.g. in Hasan Z et al (BMC immunology 2005; 6:14) diluted whole blood from controls and TB patients is stimulated with BCG and LPS and in FIG. 2 we see clear cut induction of MCP1 with time, whereas in example 5 and 6 WO 2008/064684 demonstrate no significant MCP-1 responses to LPS and BCG stimulation in the dried blood spots compared to reference.

IP-10 is a biomarker expressed when stimulating cells of the immune system with a suitable antigen or mitogen. In light of the teachings of Mei et al and the obvious inconsistency between experimental data presented on dried blood spots in WO 2008/064684 and numerous publications on CMI responses in plasma (e.g. Brock et al, Sanchez et al and Hazan et al.) it was surprising to find that IP-10 eluded from plasma and blood dried on filter paper is a reproducible, reliable and safe method to detect infection with M. tuberculosis and cytomegalovirus despite the known instability problems in the field of biomarker with relation to cell mediated immune (CMI) responses.

### SUMMARY

This disclosure describes the surprising finding that it is possible to extract useful clinical information regarding cell mediated immune response from dried plasma and blood spots on filter paper. This disclosure describes a method that enables centralization and high throughput screening of cell mediated immune responses e.g. used for the diagnosis of infection with M. Tuberculosis.

One aspect of the present invention relates to a method for measuring an antigen specific cell-mediated immune response comprising the steps of
a) incubating a sample comprising T-cells obtained from a mammal with at least one antigen
b) applying a fraction of the sample on filter paper
c) determining the level of IP-10 in a fraction of said filter paper, and
d) comparing said determined level of IP-10 in said filter paper with a reference level, thereby determining whether the mammal has previously encountered the first antigen generating immunological reactivity to the first antigen or previously encountered other antigens generating immunological cross reactivity to the first antigen.

### DETAILED DESCRIPTION

The present disclosure provides a method that enables simpler, easier and more accurate determination cell mediated immune (CMI) responses. The method use a diagnostic and prognostication assay based on measuring immune effector molecule production by cells of the immune system in response to antigenic stimulation together with a simple and safe “dried blood spot” filter paper method of storing and shipping samples. The biomarker(s) of the present invention may be detected using ligands such as antibodies specific for the effectors or by measuring the level of expression of genes encoding the effectors.

The present disclosure provides, therefore, means to determine the responsiveness of CMI in a subject and, in turn, provides means for the diagnosis of infectious diseases, pathological conditions, level of immunocompetence and a marker of T-cell responsiveness to endogenous or exogenous antigens.

One aspect of the disclosure describes a simple and fast method for retrieving the clinical information from the biomarkers of the present invention using the one-step direct biomarker detection and elution from filter paper. This is exemplified using IP-10.

The disclosure relates to an assay of the potential or capacity of a subject to mount e.g. an IP-10 response. The assay is based on measuring e.g IP-10 production by cells of the immune system in response to antigenic stimulation. The IP-10 production may be detected using ligands such as antibodies specific for IP-10 or by measuring the level of expression of genes encoding IP-10 or similar means. The present inventors here demonstrate that a test based on MTB specific stimulation, filter paper storage and subsequent determination of IP-10 can identify persons infected with M. tuberculosis.

The test system is as specific as and more sensitive than tests based on alternative biomarkers IFN-γ as effect parameter. IFN-γ levels in plasma are low and it is not possible to detect this biomarker with sufficient sensitivity using the method described herein. The test system is based on biomarker detection using an immunoassay (i.e. ELISA, Luminex or chemiluminescence) and the test system can potentially be developed into a field friendly test applicable in low resource settings, where the test results are sent by normal mail to a central lab.

The assay described in the present invention solves a series of problems. As dried spots of plasma are safe and simple to transport over long distances this disclosure enables centralization and automation of the test procedure which in return reduces cost and improves accuracy.

### The Assay

Thus, one aspect of the present invention relates to a method for measuring an antigen specific cell-mediated immune response comprising the steps of
a) incubating a sample obtained from a mammal with at least one antigen
b) applying a fraction of the sample on filter paper
c) determining the level of at least one biomarker in said filter paper
d) comparing said determined level of the at least one biomarker in said filter paper sample with a reference level, thereby determining whether the mammal has previously encountered the first antigen generating immunological reactivity to the first antigen or previously encountered other antigens generating immunological cross reactivity to the first antigen.

More specifically the sample is divided into at least 2 fractions and
a) incubating the first fraction of the sample with the antigen to generate a response sample
b) incubating the second fraction of the sample with an inactive solution to generate a nil sample
c) applying a fraction of the response sample on filter paper
d) applying a fraction of the nil sample on filter paper
e) determining the antigen dependent biomarker response of the sample by subtracting the biomarker level determined in the filter paper from the nil sample from the biomarker level determined in the filter paper from the response sample
f) comparing the antigen dependent biomarker response or a value derived thereof with the reference level or a value derived thereof, thereby determining whether mammal has previously encountered the first antigen and thus generate immunological reactivity to the first antigen or previously encountered other antigens generating immunological cross reactivity to the antigen.

### The Filter Paper

The filter paper of the present invention relates to any type of semi-permeable paper known to the skilled addressee. These filter papers are made in a varieties of ways since specific applications require specific types of papers and some non-limiting examples are: Whatman 903 (Whatman, USA), Whatman 3MM (Whatman, USA), normal toilet paper (Irma, Copenhagen), unbleached newspaper paper (Politiken, Copenhagen) and the filter paper used in the national PKU test supplied from SSI Copenhagen (SSI, Denmark).

In a preferred embodiment the sample is dried on a standardized filter paper such as but not limited to Whatman 903, Whatman 3MM. Other preferred embodiments use cellulose based paper or synthetic porous materials or glass based porous material. Cotton, Q-Tips and other means can also be used.

In a preferred embodiment the filter paper is dried until completely dry, at room temperature this is achieved from 2 to 6 hours, faster drying time is possible with higher temperature or lower humidity. A low humidity is best.

### Diagnosis

In one embodiment, the method may be used for diagnosis of subjects suspected of various immunological states, such as infections. When used in diagnosis the method according to the present invention may help to determine the presence of immunological states, such as infections, usually accomplished by evaluating clinical symptoms and further laboratory tests. The test may diagnose various stages of infection i.e. a recently encountered infection in an individual without any symptoms, an infection encountered many years back in an individual with no symptoms of that infection, an active infection where the patients has symptoms due to the infection.

In a preferred embodiment the method is useful for the diagnosis of Tuberculosis.

In another preferred embodiment the method is useful for the diagnosis of CMV infection.

It would be evident to the skilled addressee that the any infection or immunological state that can be measured by the use of the diagnostic power of IP-10, should be applicable for the present invention.

### Prognosis

In one embodiment, the method may be used for predicting the prognosis of subjects diagnosed with various immunological states, such as infections. When used in patient prognosis the method according to the present invention may help to predict the course and probable outcome of the immunological states, such as infections, thus assisting the skilled artisan in selecting the appropriate treatment method and predict the effect of a certain treatment for the condition.

In line with the described embodiments of prognosis and diagnosis the method is useful for monitoring, screening, contact tracing, prevalence studies and research purposes and it should be understood that any feature and/or aspect discussed herein in connection with the determination according to the invention apply by analogy to the “diagnosis”, “prognosis”, “monitoring”, “screening”, “research purposes”, “contact tracing”, “enhanced case finding” and “prevalence studies” according to the disclosure.

### Incubation Step

The cells of the immune system lose the capacity to mount a CMI response in whole blood after extended periods following blood draw from the subject, and responses without intervention are often severely reduced or absent 24 hours following blood draw, if not treated in a manner that prolongs there life of the cells such as, but not limited to, preservation at a temperature above 10° Celsius.

The incubation step may be from 5 to 144 hours, more preferably 5 to 120 hours and even more preferably 12 to 48 hours or a time period in between.

Incubation is preferably done at physiological temperatures. In a preferred embodiment the incubation temperature is 37° C., in another preferred embodiment the incubation temperature is 37.5° C., in another preferred embodiment the incubation temperature is 38° C., in another preferred embodiment the incubation temperature is 38.5° C., in another preferred embodiment the incubation temperature is 39° C., in another preferred embodiment the incubation temperature is 39.5° C., in another preferred embodiment the incubation temperature is 40° C., in another preferred embodiment the incubation temperature is 40.5° C., in another preferred embodiment the incubation temperature is 41° C.

The incubating step can be performed at a not fixed temperature between, but not limited to, 15° to 40° Celsius, more preferably from 37°-41° Celsius.

In one embodiment the incubation is done at a temperature from 37° C. to 39.5° C.

In another embodiment the incubation is done at 39° C.

One embodiment of the disclosure allows stimulation of sample to be performed with addition of culture media to the cell culture. Or addition of a nutrient such as simple sugar, complex sugar, disaccharides, glycan's, amino acids (e.g. glutamate) or other.

Another embodiment of the disclosure allows stimulation of sample to be performed with addition of inert dilution liquid (e.g. saline) to the cell culture.

Another embodiment of the disclosure allows removing or inhibiting cells e.g. granulocytes, regulatory T cells, Th2 cells.

The present invention also relates to improving immunodiagnostic tests by addition of at least one immune modulator during incubation. The term immune modulator is to be understood as a substance that alters the immune response. Immune modulators are substances that are able to induce adjustment of the immune response to a desired level, as in immunopotentiation, immunosuppression, or induction of immunological tolerance and/or are able to counteract potential harmful effects as cell stress due to hyperthermia. An immune modulator is also understood as a substance that is able to boost or inhibit specific areas of the immune system e.g. the T Lymphocytes cells or T lymphocyte subpopulations. Preferred immune modulators according to the present invention are cytokines and neutralizing antibodies which improve the test-antigen specific cell-mediated immune response. Particular preferred immune modulators are cytokines IL-7, IL-15, IL-21, neutralizing antibodies binding IL-10, IL-4, IL-5, beads coated with anti-CD25 antibodies, beads coated with anti-CD39 antibodies, sense or antisense oligonucleotide to genetic material encoding IL-10, JAKI or TYK2, a CpG containing oligonucleotide, an oligonucleotide acting as a TLR modulating agent, and a TLR modulating agent is added in step b. In another preferred embodiment the at least one immune modulator is a neutralizing antibody selected from the group consisting of the neutralizing antibodies binding IL-10, neutralizing antibodies binding IL-4, neutralizing antibodies binding IL-5 and neutralizing antibodies binding CD25. In yet another preferred embodiment the at least one immune modulator is selected from the group consisting of beads coated with anti-CD25 antibodies, sense or antisense oligonucleotide to genetic material encoding IL-10, JAKI or TYK2, a CpG containing oligonucleotide, an oligonucleotide acting as a TLR modulating agent, and a TLR modulating agent.

It is well established that the cytokine Interleukin-7 (IL-7) is essential for survival and homeostasis of naïve and memory CD4+ and CD8+ T-cell subsets.

Another embodiment of the disclosure allows blocking of anti-inflammatory cytokines such as IL-10, IL-4, IL-13, TGF-b and or blocking of inhibitory receptors such as PD-1.

### Sample

In one embodiment of the present disclosure contemplates a method for measuring a CMI response in a subject, said method comprising collecting a sample from said subject wherein said sample comprises cells of the immune system, which are capable of producing immune effector molecules following stimulation by an antigen, incubating said sample with an antigen and then drying the cell culture supernatant on filter paper and measuring the presence of or elevation in the level of an immune effector molecule in the filter paper wherein the presence or level of said immune effector molecule is indicative of the capacity of said subject to mount a cell-mediated immune response.

Conveniently, when the sample is whole blood, the blood collection tube is heparinised. Notwithstanding that whole blood is the preferred and most convenient sample, the present invention extends to other samples containing immune cells such as but not limited to ascites fluid, lymph fluid, spinal or cerebral fluid, tissue fluid and respiratory fluid including nasal, and pulmonary fluid.

In another embodiment the present invention thus relates to a method, wherein the sample comprises cells selected from the group consisting of whole blood, peripheral mononuclear cells, T cells, CD4 T cells, CD8 T cells, gamma-delta T cells, monocytes, macrophages and NK cells

In one embodiment, the sample is whole blood, which may be collected in three suitable containers in which, antigen, mitogen or “nil” are present or antigens, mitogen or “nil” can be added to aliquots of the whole blood afterwards.

In another embodiment, the sample is whole blood which may be collected in collection tubes containing the antigen, mitogen or “nil” or to aliquots of whole blood to which antigen, mitogen or nil is added.

In one embodiment the sample is whole blood, plasma or any other blood derived components.

### Antigen

The choice of antigen suitable for the present invention, also referred to as test-antigen(s) disease-specific antigens, and antigen selected for evaluation, depends on the type of infection the skilled addressee would like to assess, accordingly the selected antigens are disease associated. For example when monitoring MTB infection any available MTB antigens could generate the necessary response and vice versa. Several antigens are already used in the existing commercial assays. It should be understood that any feature and/or aspect discussed above or below in connection with the test-antigen(s) according to the invention apply by analogy to the antigen selected for evaluation.

Thus, the antigens of the present invention may be specific for a bacterium, a virus, e.g. specific for Mycobacteria, such as Mycobacterium tuberculosis.

Wherein the infection is believed to be related to tuberculosis, the antigen or the at least one antigen is selected from the group consisting of RD-1 antigens, ESAT-6, CFP10, TB7.7, Ag 85, HSP-65, Ag85A, Ag85B, MPT51, MPT64, TB10.4, Mtb8.4, hspX, Mtb12, Mtb9.9, Mtb32A, PstS-1, PstS-2, PstS-3, MPT63, Mtb39, Mtb41, MPT83, 71-kDa, PPE68 and LppX.

In a presently preferred embodiment the antigen or the at least one antigen is selected from the group consisting RD-11 antigens, RD-2 antigens, TBCELLSAT, ESAT-6, CFP-10, TB 7.7, Ag 85, Rv1985c, Rv3615c, HSP 65 and RD-1 antigens.

Especially relevant for this invention are antigens, which are encoded by genes present in M. tuberculosis, but not in Bacillus Calmette G (BCG). These antigens include RD-1 antigens. Another relevant set of antigens the extracellular expression of which depends on proteins that have been deleted in BCG such as the Esx-1 transporter. These antigens include Rv3615c.

In yet an embodiment of the present invention the antigen is ESAT-6.

In another embodiment of the present invention the antigen is CFP-10.

In another embodiment of the present invention the antigen is Rv3615c

In a further embodiment of the present invention the antigen is TB 7.7.

In a presently preferred embodiment of the present invention the antigens are RD-1 antigens.

In yet preferred embodiments the antigens are smaller fractions e,g, peptides from the antigen. One example of a fraction of an antigen is 15-mer peptides from the c-terminal of Rv3615c

Several research institutions are working on identification of antigens solemnly expressed by the individual infectious agent, so called microbe- or disease-specific antigens. In the case of M. tuberculosis, specific antigens are expressed at different stages of infection such as but not limited to dormant, latent, active, recent, pulmonary, extrapulmonary, localized or cured stages.

The present invention can be implemented using such antigens thus providing a tool for identification of that specific stage (e.g. latent infection with M. tuberculosis).

In a preferred embodiment, several antigens from the same microorganism can be added when generating the response sample. By adding several antigens with various tissue type preferences the strength of the assay is increased. In the case of tuberculosis, combining antigen-peptides of ESAT-6, CFP-10 and TB7.7 proteins increases the probability that the test covers the broadest range of tissue types and thus gives stronger and more reliable test results in different patient populations.

Wherein the infection is believed to be related to Chlamydia, the antigen or the at least one antigen is selected from the group consisting Serovar D extract, major outer membrane protein (MOMP), cysteine-rich outer membrane proteins (OMPs), OMP2, OMP3, Poly-morphic OMPs (POMPs), adenosine diphosphate/adenosine triphosphate translocase of Chlamydia pneumonia, porin B proteins (PorBs), and CT521.

As apparent from the present invention the source of infection may vary. In an embodiment of the present invention the antigen or the at least one antigen is selected from the group consisting of fixed-epimastigotes, fixed-trypomastigotes, disrupted-epimastigotes, disrupted-trypomastigotes, purified antigenic fractions from epimastigotes, semipurified antigenic fractions from epimastigotes, trypomastigote excretory-secretory antigens (TESA), predominant variable antigen type (VAT), variable surface glycoprotein (VSG), trans-sialidase (TS) e.g. TS13, amastigote surface protein-2 (ASP2), KMP-11m, CRA, Ag30, JL8, TCR27, Ag1, JL7, H49, TCR39, PEP-2, Ag36, JL9, MAP, SAPA, TCNA, Ag13, TcD, B12, TcE, JL5, A13, 1F8, Tc-24, Tc-28, Tc-40, Cy-hsp70, MR-HSP70, Grp-hsp78, CEA, CRP, SA85-1.1, FCaBP (flagellar Ca2+-binding protein), FL-160 (flagellar surface protein of 160 kDa) and, FRA (flagellar repetitive antigen) said antigens being related to Trypanosomas.

In a preferred embodiment of the present invention the antigen or the at least one antigen is selected from the group consisting of fixed-epimastigotes, fixed-trypomastigotes, disrupted-epimastigotes, disrupted-trypomastigotes, purified antigenic fractions from epimastigotes, semipurified antigenic fractions from epimastigotes, trypomastigote excretory-secretory antigens (TESA), predominant variable antigen type (VAT), variable surface glycoprotein (VSG), trans-sialidase (TS) e.g. TS13, amastigote surface protein-2 (ASP2), FCaBP (flagellar Ca2+-binding protein), FL-160 (flagellar surface protein of 160 kDa) and FRA (flagellar repetitive antigen).

In the case wherein the infection is related to schistosoma, the antigen or at least one antigen is selected from the group consisting of disrupted schistosoma egg, excreted/secreted glycoproteins (ES), tegumental (TG) glycoproteins, soluble egg antigen (SEA), soluble extract of S. mansoni (SWAP), keyhole limpet haemocyanin (KLH), RP26, Sj 31, Sj 32, paramyosin, Sm62-IrV5, Sm37-SG3PDH, Sm28-GST, Sm14-FABP, PR52-filamin PL45-phosphoglycerate kinase, PN18-cyclophilin, MAP3, Sm23, MAP4, Sm28-TPI, Sm97, CAA, CCA and, Schistosoma mansoni heat shock protein 70.

In a preferred embodiment of the present invention the antigen or the at least one antigen is selected from the group consisting of excreted/secreted glycoproteins (ES), tegumental (TG) glycoproteins, soluble egg antigen (SEA), soluble extract of S. mansoni (SWAP), keyhole limpet haemocyanin (KLH) and, RP26.

In respect of leishmania, the antigen or at least one antigen is selected from the group consisting of disrupted promastigozyes, leishmanin, rGBP, rORFF, rgp63, rK9, rK26, rK39, PN18-cyclophilin, MAP3, Sm23, MAP4, Sm28-TPI, Sm97, CAA and, CCA.

In fact any antigen specific for the species to be analysed could be useful according to the present invention.

It is to be understood that the term “antigen” is not restricted to a whole e.g. protein, the term also includes single peptides, overlapping peptides from a whole protein or the whole protein.

In another preferred embodiment, a range of different antigens from different diseases can be combined to enable a screening tool with low specificity for the individual disease, but high sensitivity for “infection”. A kit combining e.g. a palette of antigens from microbes soldiers are exposed to during mission (e.g. malaria, tuberculosis, leishmania, schistosoma and/or trypanosomiasis) will enable doctors to perform one quick screening-test instead of a range of different tests.

In another preferred embodiment, combined kits may comprise of antigens from various microbes infecting an organ (e.g. Nesseria and Chlamydia species causing pelvic inflammatory disease), or comprise of antigens from infectious agents that cause common symptoms (e.g. treatable diarrhea caused by campylobacter and shigella infection, could be distinguished from untreatable diarrhoea caused by virus e.g. rotavirus).

One of the first ways of the host to respond to infection depends on the innate immune system. The innate immune system comprises a variety of innate resistance mechanisms that recognize and respond to the presence of infection by secreting a multitude of signals including IP-10. Innate immunity provides an immediate, but non-specific response to infection. The response does not increase with repeated exposure to a given pathogen and is thus independent of immunological memory.

In contrast, cell mediated immune reactivity (CMI) (or adaptive immunity) is an antigen-specific and T-cell dependent adaptive immune response. Upon a first encounter with a specific antigen there is no initial antigen-dependent cell-mediated immune response however, an immune adaptation to the specific antigen molecule is generated. The specific antigen molecule is remembered, and a following exposure will lead to a strong and fast antigen specific response. CMI is measured by a cellular response traditionally by the production of IFN-γ.

In respect of the terms “antigen”, “specific”, “specific antigen”, “specific peptides”, “disease specific antigen”, “disease specific peptide”, “test antigen” these terms denotes an antigen that fingerprints “the infecting pathogen” or “a small group of pathogens that during infection describe a specific clinical disease entity”. The specificity of an antigen is e.g. examined in cellular assays or in gene sequencing analysis. Specific antigens are specific compared to non-specific that cannot convincingly fingerprint such entities. In line herewith; antigens that can generate an innate immune response cannot be classified as specific peptide or protein test antigen.

In one embodiment the antigen is specific for a microorganism.

In another embodiment the antigen is a peptide and/or a protein and/or a panel of at least 2 peptides.

Antigens may be in the form of peptide, polypeptide or protein, carbohydrate, glycoprotein, phospholipid, phosphoprotein or phospholipoprotein or non-protein chemical agent.

As the innate immune response is independent of immunological memory LPS is not a suitable antigen for generating a specific cell-mediated immune response. Accordingly, it is not possible to determining whether a mammal is likely or unlikely to have a antigen specific cell-mediated immune response. In line herewith PPD (purified protein derivative) which is a precipitate comprising a variety of predominantly non-specific antigen is not a specific antigen. Mycobacterial PPDs is a crude, poorly defined mixture of antigens comprising both secreted and somatic proteins, most of which are shared among virtually all mycobacteria including those belonging to the M. tuberculosis complex (M. tuberculosis, M. bovis, and M. africanum), environmental nontuberculous mycobacteria (NTM), and the vaccine sub-strain M. bovis bacille Calmette-Guerin. If diagnosis is based on PPD, a large number of false-positive cases are seen after BCG vaccination and exposure to NTM as PPD contains a large number of antigens shared among different mycobacterial strains. Accordingly, PPD cannot be termed a specific antigen for M. tuberculosis. Although PPD used in the tuberculin skin test has been used in vivo to diagnose infection with M. tuberculosis it is widely known that this in vivo PPD response is not specific for M. tuberculosis for the reasons just mentioned. Attempts to use PPD in vitro for diagnosis of TB has also failed due to above reasons PPD, like LPS activates the innate immune system which, as mentioned previously, is a generic non-specific response comprising the secretion of a multitude of signals including IP-10.

In a preferred embodiment, the antigen is present in the incubation chamber when the sample is added. If present before incubation it can be in lyophilized form e.g. coated on the sides of the incubation chamber or on a small filter paper disc. Antigen present in solution improves the ease of mixing the antigen with the sample. A preferred dissolvent for the peptide is water or a stabilizing solution such as StabileZyme (Surmodics, USA). It is essential that the sample does not react unspecific when put in contact with the solvent.

### Transfer to Filter Paper

Following incubation of the sample comprising cells of the immune system a fraction of the sample is transferred to filter paper. The sample being transferred is not necessarily identical to the sample being incubated. In a preferred embodiment the sample being incubated is whole blood and the sample being transferred to filter paper is plasma. In another embodiment the incubated sample is whole blood and the transferred sample is also whole blood. In yet another embodiment the incubated sample is PBMCs and the sample being transferred to the filter paper is cell culture supernatant.

Transfer can be done directly from the incubation container e.g. a blood collection device (vaccutainer). This can be done using a pipette, a pasteur-pipette or simply by using the filter paper as lid, and turning the open end of the collection device upside down and allowing the filter paper to be filled with the sample.

In another preferred embodiment the incubation chamber is a syringe with a piston. The piston is used when filling the chamber and when adding (or transferring) the sample to the paper. The syringe system is optimal for incubation of small volumes of sample.

In a preferred embodiment the sample can be concentrated before transfer to the filter paper. Such a concentration step could be done using a sponge with a filter, or simple osmosis. Alternatively the sample can be concentrated on the filter paper by addition of a substance that increases the viscosity of the sample before addition to filter paper, or simply by adding the sample several times allowing for drying in between the application steps

As evident to the skilled addressee, then a fraction of the sample obtained from the mammal according to the present invention could be any part of the sample originally obtained from the subject in question, such as 0.01-99.99% of the original sample.

The selection of the fraction depends on the working ranges of the assay selected to measure the level of the selected biomarker. Thus, in one embodiment, the faction of the sample could be less than 100% of the original sample, such as but not limited to 99%, 95%, 90%, 85%, 80%, 75%, 70%, 65%, 60%, 55%, 50%, 45%, 40%, 35%, 30%, 25%, 20%, 15%, 10%, or 5%.

### Spotting Sample to Filter Paper

It is the size of the punch (i.e. the filter paper disc) that determines the volume of sample being analyzed; therefore the volume of sample which is added to filter paper before drying and punching can differ without affecting the result of the test. The reason for this is that the sample will penetrate the paper in a uniform way generating a spot which diameter is relative to the volume added i.e. a low volume will generate a small spot and a large volume will generate a large spot.

The composition of the sample determines the preferred volume when adding sample to the filter paper and the optimal size of the filter paper punched and used for the analysis. One factor that influences the size of the spot is the viscosity of the sample: A viscous sample e.g. whole blood makes smaller spots vis-à-vis a less viscous sample such as plasma makes a larger spot. Further the thickness and the composition of the filter paper is cardinal for the size of the spot. E.g a filter paper with a 0.2 mm thickness generates spots that are half the size, as the same volume of sample added to a 0.1 mm filter paper. Many different types of sample can be added to the filter paper e.g. serum, plasma, diluted whole blood, undiluted whole blood and antigen stimulated whole blood.

Thus, the sample volume depends both on the composition of the sample and the type of filter paper used.

Any volume between 10 μL and 500 μL will typically be added to the paper. In a preferred embodiment the volume is at least 15 μL and not more than 100 μL. If using a standard Whatman 903 filter paper then the most preferred volume ranges 20-50 μL depending on the type of sample.

In other embodiments the volume is applied with less precise methods such as e.g. 3 drops of sample from a Pasteur pipette or similar.

In a preferred embodiment the size of the spot is pre-printed on the filter paper to guide in the application and estimation of added volume of sample.

### Signal Intensity and Assay Range

All analysis platforms (including ELISA, ELISPOT, Chemoluminessence and other platforms) have an optimal range within which the precision of the assay is highest. The important determinants of the assay include affinity of antibodies specific for the biomarker, buffer composition and incubation time.

For precise determination of an analyte it is important that the analyte is present at a concentration that generates signal intensity—e.g. an optical density (OD)—that fits the working range of the assay. Using ELISA as an example, the optimal ODs for reliable readings with a standard reader (e.g. at 450 nm) are in the range of 0.02-2. Accurate assays must be calibrated so that samples with unknown concentrations utilize the full spectrum of the reader. Therefore all assays have specified the working range i.e. the range of concentrations within which the assay performs reliably. ELISAs, typically have standard curves with a linear range from 10-500 pg/ml and a lower limit of detection of 2-3 pg/ml. For the concentrations in the sample to reach this range, all assays are supplied with a set of sample preparation manuals e.g. recommendations for dilution of sample before analysis.

### Methods Available to Describe the Working Range of an Assay

The optimal range is normally described in a series of standardized experiments demonstrating the range of assay, linear part of the standard curve, the lower and upper limits of detection, the lower and upper limits of quantification and the intra and inter assay variability.

In most cases the optimal assay range is the linear part of the standard curve, in this area there is a linear relation between the analyte and the generated signal (e.g. the OD reading), in contrast in the area below and above the linear part the signal/analyte relationship follows a non-linear model. Modeling of the linear part of the standard curve can be done with simple linear regression (y=a x+b), modeling the full range of the assay requires non linear regression e.g. 4- or 5-parameter logistic curve fit.

### Problems Relating to Determining Concentrations of a Sample in the Extremes of the Working Range

As evident from above it is possible to dilute a sample with a high concentration of biomarker to optimize the signal intensity (i.e. the concentration in the sample) to the optimal part of the standard curve, i.e. the working range. Low expressed markers do not have this option, as it is not practically feasible to concentrate a sample. Therefore low expressed biomarkers such as IFN-γ are per se more difficult to detect than highly expressed biomarkers such as IP-10, therefore IFN-γ can't be diluted very much before the signals are below the linear part of the curve and below detection. IP-10 on the other hand should be diluted extensively to get within the linear part of the range.

When comparing IP-10 and IFN-γ as two biomarkers for cell mediated immune response (CMI) assay for TB diagnosis IP-10 is expressed in very high levels in plasma (1000-50,000 pg/ml) compared to IFN-γ (17.5 pg/ml-500 pg/ml). To achieve an optimal fit of the samples to the working range of an ELISA a large plasma sample (50 μL) is needed for IFN-γ detection (Quantiferon ELISA), whereas only a very small volume of plasma is needed for IP-10 detection (3 μL) (Quantikine IP-10 ELISA, RnD systems). For example a 50 μL plasma sample renders 20 pg/ml IFN-γ, diluting this e.g. ×5 the remaining 10 μL plasma left for the analysis would only result in a 4 pg/ml signal which would be practically undetectable.

The signals detected in dried blood and plasma samples from filter paper correspond to very low volumes of plasma.

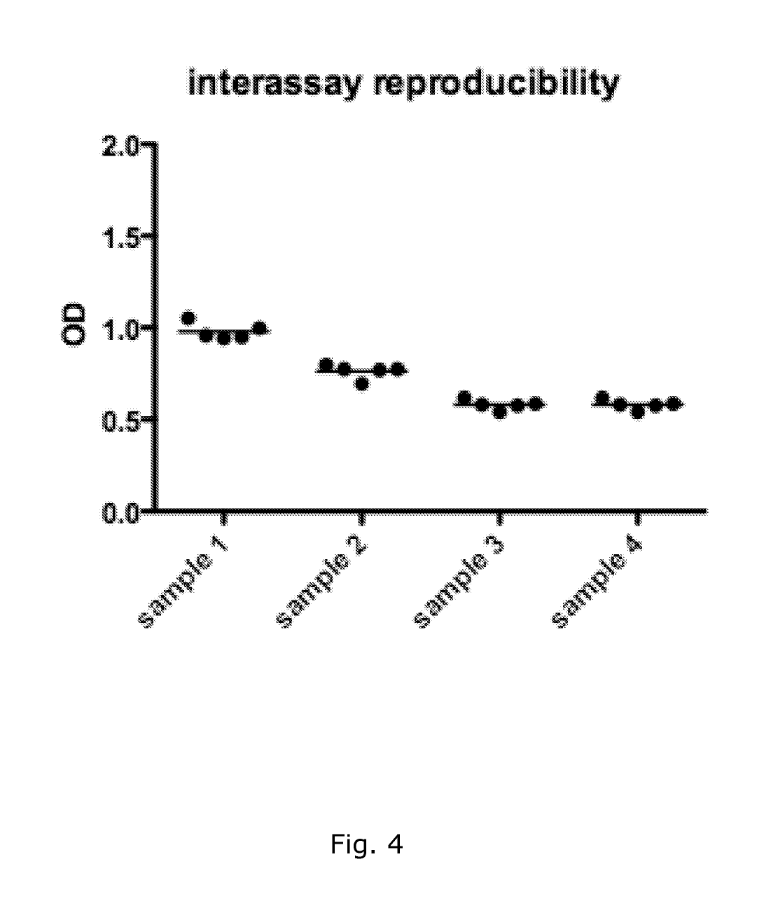

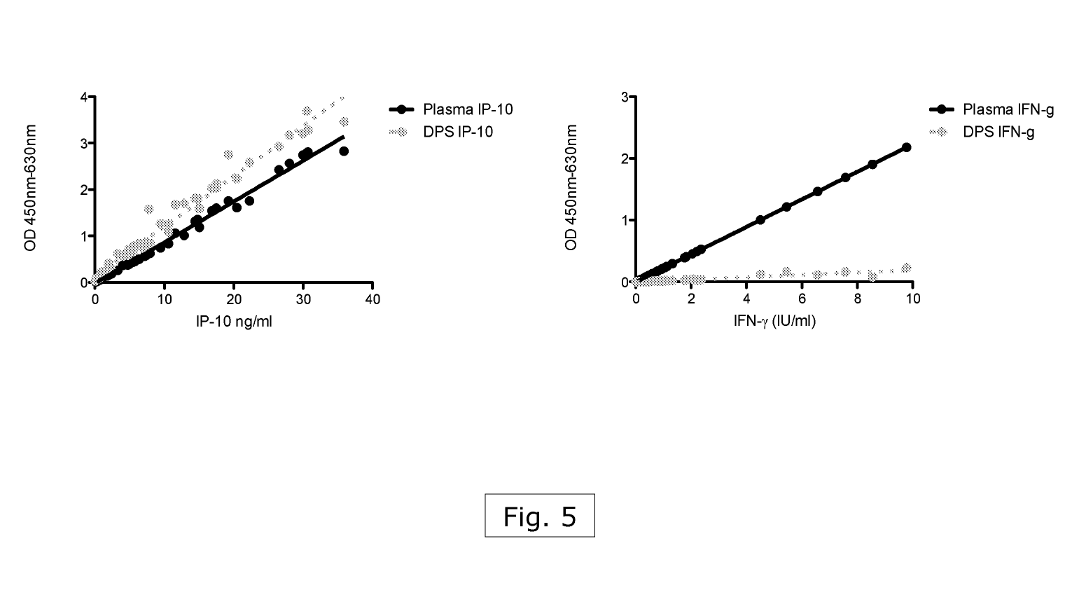

The concepts of dilution of sample and the working range of the assays are is for example illustrated in FIG. 5 where we compared the OD readings from 19 TB patients with IP-10 (left) and IFN-γ (right). The solid lines are OD readings generated in extensively diluted samples (=3 μL plasma for IP-10) and less diluted sample (=50 μL plasma for IFN-γ). These different volumes generate OD readings, which fit the respective working range of the assays very well. When we analyze the same samples in 2 spots of plasma (DPS) dotted grey lines, the OD readings for IP-10 are comparable in range as the OD readings in plasma, but the OD readings of IFN-γ fail to reach detectable levels before approx. 2 IU/ml (=100 pg/ml). In contrast to the readings of IP-10 in DPS—the signal intensity of IFN-γ in the DPS samples fall below and therefore fails to utilize the working range of the assay (0.02-2.0 OD) and as a consequence the results are very imprecise if detectable at all.

The volume of plasma or blood contained in 2 DPS or DBS discs is very low. In example 2 we have determined the volume of plasma in 2 DPS discs to 4.2 μL, and in example 9 to 4.0 μL, this volume is much lower than what is needed for a reliable readout of IFN-γ (50 μL). IP-10 is best detected in much lower volumes (e.g. 3 μL) and is therefore a strong diagnostic marker for TB in a CMI assay based on DPS or DBS samples. As illustrated in example 10 IFN-γ is a very poor candidate as the concentrations of IFN-γ detected in 2 DPS discs fails to reach reliable and even detectable levels in many of the investigated samples. In example 9 we compare the signal intensity of IP-10 in plasma, 2 discs of DBS and 2 discs of DPS. We estimate that 1 μL of plasma corresponds to 11.9 mm2 DPS/DBS given that the filter paper is Whatman 903. From this it is clear why IP-10 performs comparable in 3 μL plasma and 2 5.5 mm discs (=47.6 mm2) of DPS or DBS (in a 100 μL ELISA well). If one was to get comparable DPS or DBS signals for the standard 50 μL plasma sample volume used for IFN-γ in the Quantiferon test, then this signal would require 595 mm2 of DBS or DPS on Whatman 903 filter paper (50×11.9 mm2), corresponding to 25 discs of 5.5 mm diameter (595 mm2/23.8 mm2). This large area of paper is not feasible to spot with sample or get into the ELISA well.

It is interesting that there is only little difference in signal intensity between DPS and DBS samples. This is because plasma contains more biomarker per μL but is less viscous and makes larger spots. Blood—in contrast—is comprised of 40% cells wherefore the biomarker/μL is less than plasma but the viscosity is higher. As a consequence blood makes smaller spots on filter paper than plasma, but the spots as demonstrated in example 9 generate comparable signal levels per DBS and DPS disc.

In a preferred embodiment of the present invention, then when the determining the level of IP-10 in a fraction of said filter paper then the size of the fraction of filter paper is between 10 and 200 mm2, 12-180 mm2, 14-160 mm2, 16-140 mm2, 18-120 mm2, 20-100 mm2, 22-80 mm2, 24-60 mm2.

In other preferred embodiments the size of the filter paper is 25 mm2 or 50 mm2.

As obvious to the skilled addressee these estimated are based on Whatman 903 filter paper other types of paper contain different volumes of sample pr. mm2 wherefore these types of paper will result in other preferred fractions of said filter paper.

It should be known that the sample or fraction of the sample being transferred to filter paper can be characterized as “the sample”, “sample”, “the supernatant” and other terms known in the art. Similar the sample being incubated can be denoted “sample”, “the blood”, “the tissue” or other.

In other embodiments of the disclosure the sample being transferred is mixed with a stabilizing agent before addition to the filter paper. In a preferred embodiment the stabilizing agent is a buffer comprising protease inhibitors, detergent, pH stabilizing agents, sugars, non-reducing sugars, complex sugars, trehalose, proteins, bovine serum albumin, and/or other molecules with biomarker stabilizing abilities known to the skilled addressee. In a preferred embodiment the stabilizing agent is added to the sample after incubation, but before transfer of a fraction of the sample to the filter paper.

The present application disclose how to provide a fast but yet sensitive and specific method for measuring a cell-mediated immune response and enable optimal storage and analysis possibilities, this can be achieved by storing plasma samples on filter paper and by measuring a biomarker such as IP-10.

### Storage

In a preferred embodiment the filter paper samples are stored in low gas-permeability plastic bags with desiccant added to reduce humidity. The preferred temperature for long term storage is minus 80° C., but even prolonged storage at +5° C., ambient temperature and even in tropical climates are possible. The most important factor for the stability of the sample dried on filter paper is protection from humidity.

### Hole Punching

In a preferred embodiment the holes in the filter paper are punched with a hole punch (known also as a hole puncher, paper puncher, holing pincer, or rarely perforator), a common office tool that is used to create holes in sheets of paper. A typical hand held hole punch, whether a single or multiple hole punch, has a long lever which is used to push a bladed cylinder straight through the paper. As the vertical travel distance of the cylinder is only a few millimeters, it can be positioned within a centimeter of the lever fulcrum.

Another mechanism uses hollowed drills that are lowered by a screwing action into the paper. The paper is cut and forced up into the shaft of the drill to be later discarded. This method allows a small machine to cut industrial volumes of paper with little effort.

In another embodiment the hole punching is automated and the optimal placement of the hole puncher on the filter paper is guided using laser or other accurate tools.

In a preferred embodiment the lab technician separate a small disc of saturated paper from the sheet using an automated or manual hole punch, dropping the disc or discs into a flat bottomed microtitre plate.

In another preferred embodiment the hole punching is automated e.g. using the Wallac auto puncher (PerkinElmer, USA).

As evident to the skilled addressee, then typically only a fraction of the filter paper is used for the determination of the IP-10 level, thus according to the present invention such fraction of the filter paper could be any part of the filter paper having the originally obtained sample from the subject in question applied, such as 0.01-99.99% of the original sample.

The selection of the fraction of the filter paper again depends on the working ranges of the assay selected to measure the level of the selected biomarker. Thus, in one embodiment, the faction of the filter paper could be less than 100% of the original filter paper, such as but not limited to 99%, 95%, 90%, 85%, 80%, 75%, 70%, 65%, 60%, 55%, 50%, 45%, 40%, 35%, 30%, 25%, 20%, 15%, 10%, or 5%.

### Elution

Elution is the extraction plasma proteins such as biomarkers from the filter paper.

In one embodiment the biomarker in the sample is eluted in a buffer. This buffer could be water, saline, pH buffered saline, or saline added a detergent (e.g. tween 80, tween 20), sodium azide, bronidox, and/or bovine serum albumin.

In a preferred embodiment elution is done with a protease inhibitor added to the buffer. One example of a protease inhibitor is Sitagliptin which specifically inhibits DPP-4.

Elution can be improved with shaking, (e.g. on a mechanical shaker) of the sample, increasing the temperature or by other mechanisms.

In one embodiment the biomarker level is determined by elution of the biomarker from the filter paper in a buffer.

In one embodiment the biomarker level is determined by elution of the biomarker and detecting the biomarker in one step.

In one embodiment the combined elution and detection step is a done using pair of monoclonal antibodies binding the biomarker creating a sandwich.

In another embodiment the sandwich is coupled to an enzyme, a molecule capable of generating chemoluminescence or other means of signal amplification.

In another embodiment the sandwich binds a solid phase e.g. a plastic surface.

In a special embodiment the present invention relates to a method wherein the biomarker is IP-10, the antigen is a pool of peptides specific for mycobacterium tuberculosis, and the IP-10 level is eluted and determined using ELISA.

In a preferred embodiment the biomarker is eluted and detected in one single step. This is e.g. achieved by performing the elution in a well coated with mAbs and the buffer containing a mAb coupled with an enzyme or other substance that allows amplification of the signal.

In a preferred embodiment the elution is done directly in an ELISA plate coated with monoclonal antibodies specific for the biomarker one wishes to determine. The elution is done in a buffer comprising the detection antibody coupled to an enzyme. In another preferred embodiment the elution is done in a coated ELISA plate, after washing the detection mAb is then added. Alternatively elution is done in a buffer comprising beads that substitute the solid phase (e.g. the plastic surface of the ELISA plate) this method could be xMAP/Luminex technology but other types of beads e.g. magneticbeads. Other preferred types of beads including fluorescent or beads linked to molecules capable of chemiluminescence can be used.

### Readout of Signal

The biomarker production is determinate by any cytokine or chemokine detection method known to the skilled addressee such as but not limited to xMAP, multiplexing, Luminex, ELISA, Chemiluminesence, FLISA assays, DELFIA assays, luminescence assays, electrochemiluminescence assays, scintillation proximity assays, radioimmunoassays, MALDI-MS, ESI-MS and ambient-MS (e.g. DESI-MS). Most preferably the biomarker is determined with a sandwich mAb technique where one of the mAbs is linked to an enzyme, a chemiluminescent or other substances with the ability to generate an amplified signal. The signal can also be detected with qPCR or other means of detecting messenger RNA or other types of nucleic acids.

The quantity of biomarkers such as IP-10 in response to antigens (e.g. tuberculosis specific proteins or derivatives hereof) may be determined by subtracting the background production of biomarker and the probable infection with e.g. M. tuberculosis is interpreted on the basis of this antigen specific biomarker response.

### Biomarker

The term “biomarker” relates to e.g. a protein which is expressed in high levels with antigen stimulation and is constitutively expressed in low concentrations.

In one embodiment the biomarker is selected from the group comprising IP-10, MIG, MCP-2, MCP-1, IL-1RA, IL-2, IFN-γ, MIP-1a, MIP-1b, sIL-2R, IL-10, TNF-α.

In a preferred embodiment of the invention the biomarker is an immune signaling molecule.

In one embodiment the biomarker is a cytokine.

In one embodiment, the biomarker level is determined in a fraction or several fractions of the filter paper with a known size.

In a preferred embodiment the term biomarker comprise a combination of at least 1 individual protein. In yet another embodiment the preferred biomarker is a chemokine or at least two chemokines.

### IP-10

IFN-γ-inducible protein 10 (IP-10) or CXCL10 is a chemokine. The IP-10 gene is mapped to 4q21 by in situ hybridization. IP-10 expression is up regulated by Interferons (IFNs i.e. Interferon gamma (IFN-γ)) and inflammatory stimuli (e.g. TNF-α and toll like receptors), and it is expressed in many Th1-type inflammatory diseases in a variety of tissues and cell types.

The human gene sequence can be found under ACCESSION number BC010954 (gi 15012099) in Gene Bank.

In a preferred embodiment this biomarker is the chemokine IP-10.

In another preferred embodiment the biomarker is IP-10, which has been truncated 2 amino acids in the n-terminal.

In another preferred embodiment the biomarker is IP-10, which has not been enzymatically cleaved.

### Kit

The present disclosure further contemplates a kit for assessing a subject's capacity to mount a cell mediated immune response. The kit is conveniently in compartmental form with one or more compartments adapted to receive a sample from a subject such as whole blood purified cells, biopsies or other material. That compartment or another compartment may also be adapted to contain heparin where the sample is whole blood.

Generally, the kit is in a form which is packaged for sale with a set of instructions. The instructions would generally be in the form of a method for measuring a CMI response in a subject, said method comprising collecting a sample from said subject wherein said sample comprises cells of the immune system, which are capable of producing immune effector molecules following stimulation by an antigen, incubating said sample with an antigen supplied with kit, drying the said sample on filter paper and then measuring the presence or elevation in level of biomarker, wherein the presence or level of said immune effector molecule is indicative of the capacity of said subject to mount a cell-mediated immune response.

The assay may also be automated or semi-automated and the automated aspects may be controlled by computer software.

The assay of the present disclosure may be automated or semi-automated for high throughput screening or for screening for a number of biomarkers from the one subject. The automation is conveniently controlled by computer software and labeling using e.g. bar codes or similar. The present invention contemplates a computer program product, therefore, for assessing the presence or absence or the level of biomarker, said product comprises
(1) code that receives, as input values, the identity of a reporter molecule associated with a labeled antibody
(2) code that compares said input values with reference values to determine the level of reporter molecules and/or the identity of the molecule to which the reporter molecule is attached; and
(3) a computer readable medium that stores the codes.

Still another aspect of the present invention extends to a computer for assessing the presence or absence or level of IP-10, said computer comprises:
(1) a machine-readable data storage medium composing a data storage material encoded with machine-readable data, wherein said machine-readable data I comprise input values which identify a reporter molecule associated with a labeled antibody;
(2) a working memory for storing instructions for processing said machine-readable data,
(3) a central-processing unit coupled to said working memory and to said machine readable data storage medium, for processing said machine readable data to compare said values to provide an assessment of the identity or level of reporter molecules or of molecules to which they are attached; and
(4) an output hardware coupled to said central processing unit, for receiving the results of the comparison.
As is evident from example 5 the correlation between plasma and DPS IP-10 is perfect (r2=0.97) and that the correlation for IFN-γ is not very good (r2=0.56). The underlying reasons are that the plasma and DPS/DBS assays for IP-10 operate in the same working range and IP-10 signals extracted from the plasma and DPS/DBS samples are comparable in terms of signal strength. This is not the case for IFN-γ, as the IFN-γ assay fails to detect low responses in DPS samples and overestimates very high IP-10 responses (compared to the assay for plasma).

### Specificity and Sensitivity

The sensitivity of any given diagnostic test define the proportion of individuals with a positive response who are correctly identified or diagnosed by the test, e.g. the sensitivity is 100%, if all individuals with a given condition have a positive test. The specificity of a given screening test reflects the proportion of individuals without the condition who are correctly identified or diagnosed by the test, e.g. 100% specificity is, if all individuals without the condition have a negative test result.

Sensitivity is defined as the proportion of individuals with a given condition (e.g. active TB infection), who are correctly identified by the described methods of the invention (e.g. has a positive test result).

Specificity herein is defined as the proportion of individuals without the condition (e.g. active TB infection), who are correctly identified by the described methods of the invention (e.g. has a negative test result)

### Receiver-Operating Characteristics

Accuracy of a diagnostic test is best described by its receiver-operating characteristics (ROC) The ROC graph is a plot of all of the sensitivity/specificity pairs resulting from continuously varying the decision threshold over the entire range of data observed.

One convenient goal to quantify the diagnostic accuracy of a laboratory test is to express its performance by a single number. The most common global measure is the area under the ROC plot.

Clinical utility of the novel method may be assessed in comparison to and in combination with other diagnostic tools for the given infection. In the case of infection with M. tuberculosis clinical utility of the novel method may be assessed in comparison to established diagnostic tools using a receiver operator curve analysis.

Thus, it is an object of preferred embodiments of the present invention to provide an immunological method for detecting whether a mammal has encountered a cell mediated immune responses, the method comprising:
a) determining the level of antigen specific biomarker production in a sample applied onto a filter paper as described herein
b) constructing a percentile plot of the biomarker level obtained from a healthy population
c) constructing a ROC (receiver operating characteristics) curve based on the biomarker level determined in the healthy population and on the biomarker level determined in a population who has generated immunological reactivity to the antigen in question
d) selecting a desired specificity
e) determining from the ROC curve the sensitivity corresponding to the desired specificity
f) determining from the percentile plot the biomarker level corresponding to the determined sensitivity; and
g) predicting the individual to have immunological reactivity to the antigen, if the level of biomarker in the sample is equal to or higher than said biomarker level corresponding to the determined specificity and predicting the individual as unlikely or not to having immunological reactivity to the antigen if the level of biomarker in the sample is lower than said total biomarker level corresponding to the determined specificity.

The specificity of the method according to the present invention may be from 70% to 100%, more preferably 80% to 100%, more preferably 90% to 100%. Thus in one embodiment of the present invention the specificity of the invention is 80%, 81%, 82%, 83%, 84%, 85%, 86%, 87%, 88%, 89%, 90%, 91%, 92%, 93%, 94%, 95%, 96%, 97%, 98%, 99% or 100%.

The sensitivity of the method according to the present invention may be from 70% to 100%, more preferably 80% to 100%, more preferably 90% to 100%. Thus in one embodiment of the present invention the sensitivity of the invention is 80%, 81%, 82%, 83%, 84%, 85%, 86%, 87%, 88%, 89%, 90%, 91%, 92%, 93%, 94%, 95%, 96%, 97%, 98%, 99% or 100%.

### Cut Off Levels

As will be generally understood by those of skill in the art, methods for screening for cell-mediated immune reactivity are processes of decision making by comparison. For any decision making process, reference values based on subjects having the disease or condition of interest and/or subjects not having the disease, infection, or condition of interest are needed.

In the specific experimental setups described herein, the level threshold of IP-10 useful as a cut off value was found to be in the range of but not limited to 14 pg/ml discs to 1000 pg/ml.

In the specific experimental setups described herein, the level threshold of IP-10 useful as a cut off value was found to be in the range of but not limited to 14 pg/2 discs to 1000 pg/2 discs.

Preferably the cut off value may be 50 pg/2 discs, 51 pg/2 discs, 52 pg/2 discs, 53 pg/2 discs, 54 pg/2 discs, 55 pg/2 discs, 56 pg/2 discs, 57 pg/2 discs, 58 pg/2 discs, 59 pg/2 discs, 60 pg/2 discs, 61 pg/2 discs, 62 pg/2 discs, 63 pg/2 discs, 64 pg/2 discs, 65 pg/2 discs, 66 pg/2 discs, 67 pg/2 discs, 68 pg/2 discs, 69 pg/2 discs, 70 pg/2 discs, 71 pg/2 discs, 72 pg/2 discs, 73 pg/2 discs, 74 pg/2 discs, 75 pg/2 discs, 76 pg/2 discs, 77 pg/2 discs, 78 pg/2 discs, 79 pg/2 discs, 80 pg/2 discs, 81 pg/2 discs, 82 pg/2 discs, 83 pg/2 discs, 84 pg/2 discs, 85 pg/2 discs, 86 pg/2 discs, 87 pg/2 discs, 88 pg/2 discs, 89 pg/2 discs, 90 pg/2 discs, 91 pg/2 discs, 92 pg/2 discs, 93 pg/2 discs, 94 pg/2 discs, 95 pg/2 discs, 96 pg/2 discs, 97 pg/2 discs, 98 pg/2 discs, 99 pg/2 discs, 100 pg/2 discs, 101 pg/2 discs, 102 pg/2 discs, 103 pg/2 discs, 104 pg/2 discs, 105 pg/2 discs, 106 pg/2 discs, 107 pg/2 discs, 108 pg/2 discs, 109 pg/2 discs, 110 pg/2 discs, 111 pg/2 discs, 112 pg/2 discs, 113 pg/2 discs, 114 pg/2 discs, 115 pg/2 discs, 116 pg/2 discs, 117 pg/2 discs, 118 pg/2 discs, 119 pg/2 discs, 120 pg/2 discs, 121 pg/2 discs, 122 pg/2 discs, 123 pg/2 discs, 124 pg/2 discs, 125 pg/2 discs, 126 pg/2 discs, 127 pg/2 discs, 128 pg/2 discs, 129 pg/2 discs, 130 pg/2 discs, 131 pg/2 discs, 132 pg/2 discs, 133 pg/2 discs, 134 pg/2 discs, 135 pg/2 discs, 136 pg/2 discs, 137 pg/2 discs, 138 pg/2 discs, 139 pg/2 discs, 140 pg/2 discs, 141 pg/2 discs, 142 pg/2 discs, 143 pg/2 discs, 144 pg/2 discs, 145 pg/2 discs, 146 pg/2 discs, 147 pg/2 discs, 148 pg/2 discs, 149 pg/2 discs, 150 pg/2 discs, 151 pg/2 discs, 152 pg/2 discs, 153 pg/2 discs, 154 pg/2 discs, 155 pg/2 discs, 156 pg/2 discs, 157 pg/2 discs, 158 pg/2 discs, 159 pg/2 discs, 160 pg/2 discs, 161 pg/2 discs, 162 pg/2 discs, 163 pg/2 discs, 164 pg/2 discs, 165 pg/2 discs, 166 pg/2 discs, 167 pg/2 discs, 168 pg/2 discs, 169 pg/2 discs, 170 pg/2 discs, 171 pg/2 discs, 172 pg/2 discs, 173 pg/2 discs, 174 pg/2 discs, 175 pg/2 discs, 176 pg/2 discs, 177 pg/2 discs, 178 pg/2 discs, 179 pg/2 discs, 180 pg/2 discs, 181 pg/2 discs, 182 pg/2 discs, 183 pg/2 discs, 184 pg/2 discs, 185 pg/2 discs, 186 pg/2 discs, 187 pg/2 discs, 188 pg/2 discs, 189 pg/2 discs, 190 pg/2 discs, 191 pg/2 discs, 192 pg/2 discs, 193 pg/2 discs, 194 pg/2 discs, 195 pg/2 discs, 196 pg/2 discs, 197 pg/2 discs, 198 pg/2 discs, 199 pg/2 discs, 200 pg/2 discs, 201 pg/2 discs, 202 pg/2 discs, 203 pg/2 discs, 204 pg/2 discs, 205 pg/2 discs, 206 pg/2 discs, 207 pg/2 discs, 208 pg/2 discs, 209 pg/2 discs, 210 pg/2 discs, 211 pg/2 discs, 212 pg/2 discs, 213 pg/2 discs, 214 pg/2 discs, 215 pg/2 discs, 216 pg/2 discs, 217 pg/2 discs, 218 pg/2 discs, 219 pg/2 discs, 220 pg/2 discs, 221 pg/2 discs, 222 pg/2 discs, 223 pg/2 discs, 224 pg/2 discs, 225 pg/2 discs, 226 pg/2 discs, 227 pg/2 discs, 228 pg/2 discs, 229 pg/2 discs, 230 pg/2 discs, 231 pg/2 discs, 232 pg/2 discs, 233 pg/2 discs, 234 pg/2 discs, 235 pg/2 discs, 236 pg/2 discs, 237 pg/2 discs, 238 pg/2 discs, 239 pg/2 discs, 240 pg/2 discs, 241 pg/2 discs, 242 pg/2 discs, 243 pg/2 discs, 244 pg/2 discs, 245 pg/2 discs, 246 pg/2 discs, 247 pg/2 discs, 248 pg/2 discs, 249 pg/2 discs, 250 pg/2 discs or as evident when stated in ml.

Other types of filter paper than Whatman 903 will generate different cut offs depending on the thickness of the paper. Vis-à-vis would samples with different viscosity than whole blood or plasma.

### Mammal

Reference to a “mammal” or a “subject” includes a human or non-human species including primates, livestock animals such as but not limited to sheep, cows, pigs, horses, donkey, goats, laboratory test animals and companion animals. The present invention has applicability, therefore, in human medicine as well as having livestock and veterinary and wild life applications.

### Microorganism

In a presently preferred embodiment the microorganism is selected from the group consisting of Mycobacteria, gram positive bacteria, gram negative bacteria, Listeria, enterococci, Neisseria, vibrio, treponema (Syphilis), Borrelia, leptospiræ, Clamydia, retroviruses (SIV, HIV-1, HIV-2), Cytomegalovirus, poxviruses, Ebstein barr virus, enterovirus, morbillivirus, rhabdoviruses (rabies). Rubivirus (rubella), flaviviruses (dengue, yellow fever), herpes viruses, varicella-zoster virus, Hepatitis C and B, Leishmania, Toxoplasma gondii, trypanosoma, Plasmodium (falciparum, vivax, ovale, malaria), pneumocystis cariini (PCP), Coronavirus (e.g. Severe Acquired Respiratory Syndrome (SARS)), Ebola or Marburg or various nematodes, trematodes

Mycobacteria belongs to the M. tuberculosis complex organisms (M. tuberculosis, M. bovis and M. africanum), and Mycobacteria where the region of difference (RD1) has not been deleted (M. kansasii, M. szulgai, M. marinum, M. flavescens, M. gastrii) or Mycobacteria pathogenic to humans (M. avium and M. lepra) or other non-tuberculous mycobacteria.

Thus, in one presently preferred embodiment the Mycobacteria is M. tuberculosis

### Vaccination

One aspect of the present invention relates to a method, wherein the antigen dependent biomarker response above the reference level indicate that the mammal has previously encountered the antigen or previously encountered other antigens generating cross reactivity to the antigen because of a vaccination against any micro-organism mentioned herein.

### Tuberculosis

Tuberculosis (commonly abbreviated as TB) is an infectious disease caused by the bacterium Mycobacterium tuberculosis, which most commonly affects the lungs (pulmonary TB) but can also affect all other organs in the body e.g. the central nervous system (meningitis), lymphatic system, circulatory system (miliary tuberculosis), genitourinary system, bones and joints. Infection with M. tuberculosis can also remain asymptomatic a stage which is commonly known as latent, dormant or sub-clinical TB infection.

In a presently preferred embodiment, the present invention relates to a method of diagnosing and monitoring various e.g. distinct presentations of tuberculosis: active tuberculosis disease, active microscopy positive or microscopy negative TB infection, latent tuberculosis infection, and recent tuberculosis infection.

The immune assay is based on the evaluation of the production of biomarker by antigen-specific T lymphocytes responding to selected peptide sequences of secretory proteins of MTB These peptide sequences have been selected for their immunogenicity and their specificity, and potentially other peptides can be used similarly.

The method and the kit can be used for diagnosing active tuberculosis disease, for diagnosing a recent infection in healthy contacts of a patient with a sputum-positive pulmonary tuberculosis, for diagnosing healthy with latent infection, for monitoring the response to treatment in the case of pulmonary and extra-pulmonary tuberculosis and to discriminate between latent infection and active tuberculosis disease state

### General Aspects of the Invention

As will be apparent, preferred features and characteristics of one aspect of the invention may be applicable to other aspects of the invention. The invention may be embodied in other specific forms without departing from the spirit or essential characteristics thereof. The foregoing embodiments are therefore to be considered in all respects illustrative rather than limiting on the invention described herein. Scope of the invention is thus indicated be the appended claims rather than by the foregoing description, and all changes that come within the meaning and range of equivalency of the claims are intended to be embraced by reference therein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention will hereinafter be described by way of the following non-limiting Figures and Examples.

### EXAMPLES

### General Method

### Stimulation

Whole blood was stimulated with disease specific antigens such as e.g. (proteins and/or peptides), lipopolysaccharide (LPS), or phytohaemaglutinin (PHA) for 18 hours at 37 degrees Celsius in an Eppendorph tube, a Vaccutainer tube or a culture plate. For the TB diagnostic studies we used the Quantiferon in tube tubes. A test system comprising three vacationer tubes: the nil tube with contains a few μL saline, the antigen tube comprising overlapping peptides from the M. tuberculosis specific antigens ESAT-6, CFP10 and TB10.4, the third tube comprising PHA mitogen. In some experiments we used the Quantiferon CMV tubes, these are comparable to the Quantiferon TB tubes in design but differ in the peptides used.

### Transfer to Filter Paper

Following incubation, whole blood or blood plasma was placed on filter paper in 10-50 μL spots (most often 25 μL) either using a pipette or a capillary tube to make the dried plasma spots (DPS) or dried blood spots (DBS). After 10 minutes to 24 hours drying at room temperature, the filter paper was stored at various temperatures (−80 to +50 degrees Celsius) and various degrees of humidity for 1 minute to 4 months.

For these experiment we used various types of filter paper: Whatman 903 (Whatman, USA), Whatman 3MM (Whatman, USA), normal toilet paper (Irma, Copenhagen), unbleached news paper paper (Politiken, Copenhagen) and the filter paper used in the national PKU test supplied from SSI Copenhagen (SSI, Denmark).

### Hole Punch

Discs were cut from the filter paper using a normal (IS0838) stationary 5.5 mm hole punch (Bantex 9303, Bog og Idé, Copenhagen), a pair of scissors (Fiskars, Finland) or a 3 mm single hole punch (Millipore, USA).

### Biomarker Determination

The optimal method for generating strong, reproducible and faster results was the optimized direct elution and incubation method. In this method the discs were placed directly in the coated ELISA wells in a buffer containing the secondary-HRP conjugated mAb (detection mAb). In some of the examples we extracted the biomarker from the discs in the buffer for 1 to 24 hours where after the buffer was transferred without the discs to the ELISA plate and incubated as described, this approach took longer time and was more laborious. Incubation was done for 2 hours, but in some experiments from 30 min to 6 hours. The preferred buffer was a PBS buffer with 2% bovine serum albumin and 0.1% Tween-20. Following 1 to 4 washes with PBS 0.1% Tween-20, 100 μL TMB substrate was added. The plate was placed dark at room temperature for 30 minutes where after the reaction was stopped by the addition of 100 μL H₂SO₄ and the optical density (OD) was read using standard ELISA reader equipment at 450 nm (ELX50 Biotek, USA). Biomarker concentration measured in the filter paper disc was determined by comparison to samples with known concentration using linear regression (standard curve).

### Example 1

### Diagnosing Tuberculosis

3×1 ml of whole blood from 36 healthy controls never exposed to TB and whole blood from 67 patients with culture confirmed active tuberculosis was incubated at 37° C. for 18 hours in the Quantiferon in Tube system, 2×25 μL plasma was transferred to Whatman 903 paper and dried for 4 hours before storage at 4 weeks at 20° C. in sealed plastic bag with a desiccator as described in the General Method above. 2 discs were cut using the 5.5 mm standard hole punch and analyzed using the direct extraction and incubation method. In FIG. 1 we present the antigen-dependent IP-10 values. In FIG. 2 the same samples are presented in a ROC curve analysis.

### Results

IP-10 extracted from dried blood spots using the direct extraction and incubation method comprises a very useful diagnostic system at par with the ELISA methods using normal plasma and IP-10 or IFN-γ measurements. 79% sensitivity at 100% specificity.

### Example 2

Using IP-10 levels to determine the volume of plasma that corresponds to the signal extracted from 1, 2, and 3 discs of dried plasma. Whole blood was stimulated with LPS for 18 h plasma was harvested. Plasma spots of 25 μL were blotted on Whatman 903, dried, punched with a standard 5.5 mm puncher and the recovery from 1, 2 and 3 discs/well (in duplicates) was compared to 1-10 μL of the identical plasma analyzed with ELISA. The graph illustrates one representative example of 4 parallel experiments done.

### Results

The mean recovery in 1, 2, and 3 discs were 1.4 μL, 4.2 μL and 6.8 μL.

### Example 3

### Reproducibility of 4 Donors

Whole blood from 4 different donors was stimulated with LPS for 18 h, plasma was harvested. Spots of 25 μL were blotted on Whatman 903, dried, and placed in sealed plastic bags with desiccant for 2 weeks. Results from 5 parallel wells of 2 discs/well are presented.

### Result

The methods has a inter assay CV %<15%.

### Example 4

### IP-10 Stability Dried on Filter Paper

Whole blood from three donors was stimulated with LPS for 18 h, plasma was harvested. Spots of 25 μL were blotted on Whatman 903, dried, and placed in sealed plastic bags with desiccant and exposed to different temperatures (5 C, 23 C, 37 C and 50 C) for up to 4 weeks. After exposure to heat, samples were stored at −20 for at least 4 weeks. 2 discs were cut using the 5.5 mm standard hole punch and analyzed using the direct extraction and incubation method.

### Results

Results table 1-4: This un-optimized system enables stabile storage of DPS samples for at least 4 weeks at (at least) 37° C. Similar results were obtained for whole blood (data not shown).

TABLE 1
+5° C.

| | Conc. | | Conc. | | Conc. | |
|---|---|---|---|---|---|---|
| | (pg/2 | Recovery | (pg/2 | Recovery | (pg/2 | Recovery |
| | discs) | (%) | discs) | (%) | discs) | (%) |
| | Sample 1 | | Sample 2 | | Sample 3 | |
| 0 w | 313 | 100 | 269 | 100 | 329 | 100 |
| 1 w | 289 | 92 | 231 | 86 | 367 | 112 |
| 2 w | 279 | 89 | 217 | 81 | 346 | 105 |
| 4 w | 272 | 87 | N/A | N/A | 318 | 97 |

TABLE 2
+23° C.

| | Conc. | | Conc. | | Conc. | |
|---|---|---|---|---|---|---|
| | (pg/2 | Recovery | (pg/2 | Recovery | (pg/2 | Recovery |
| | discs) | (%) | discs) | (%) | discs) | (%) |
| | Sample 1 | | Sample 2 | | Sample 3 | |
| 0 w | 313 | 100 | 269 | 100 | 329 | 100 |
| 1 w | 328 | 105 | 284 | 106 | 322 | 98 |
| 2 w | 335 | 107 | 293 | 109 | 346 | 105 |
| 4 w | 341 | 109 | N/A | N/A | 292 | 89 |

TABLE 3
+37° C.

| | Conc. | | Conc. | | Conc. | |
|---|---|---|---|---|---|---|
| | (pg/2 | Recovery | (pg/2 | Recovery | (pg/2 | Recovery |
| | discs) | (%) | discs) | (%) | discs) | (%) |
| | Sample 1 | | Sample 2 | | Sample 3 | |
| 0 w | 313 | 100 | 269 | 100 | 329 | 100 |
| 1 w | 350 | 112 | 299 | 111 | 343 | 104 |
| 2 w | 285 | 91 | 284 | 106 | 314 | 96 |
| 4 w | 264 | 84 | N/A | N/A | 283 | 86 |

TABLE 4
+50° C.

| | Conc. | | Conc. | | Conc. | |
|---|---|---|---|---|---|---|
| | (pg/2 | Recovery | (pg/2 | Recover | (pg/2 | Recovery |
| | discs) | (%) | discs) | (%) | discs) | (%) |
| | Sample 1 | | Sample 2 | | Sample 3 | |
| 0 w | 313 | 100 | 269 | 100 | 329 | 100 |
| 1 w | 313 | 100 | 277 | 103 | 291 | 89 |
| 2 w | 228 | 73 | 238 | 89 | 239 | 73 |
| 4 w | 216 | 69 | N/A | N/A | 223 | 68 |

### Example 5

### Benchmark DPS Technology IP-10 Versus INF-γ

3×1 ml of whole blood from 18 tuberculosis patients was incubated at 37° C. for 18 hours in the Quantiferon in Tube system, 2×25 μL plasma was transferred to Whatman 903 paper and dried for 4 hours before storage at 2 weeks at 20° C. in sealed plastic bag with a desiccator as described in the General Method above. 2 sets of 2 discs were cut using the 5.5 mm standard hole punch and analyzed using the direct extraction and incubation method using both the IP-10 ELISA and a sensitive ELISA for IFN-γ detection (CMI, Cellestis, Australia). IFN-γ was determined in plasma using the recommendations from the manufacturer' i.e. 50 μL plasma per sample well.

In FIG. 5 we present the optical density (OD) measurements obtained from plasma samples (black spots and solid line) and DPS samples (grey dots and grey dotted line) plotted against the levels measured in plasma (x-axis).

Results: IP-10 generates comparable OD readings in both plasma and filter paper samples, IFN-γ levels are well within range for the plasma samples, but very low for the DPS samples and failing to reach a reliable signal intensity. This example demonstrates that IP-10 is detectable in DPS system; one marker, which seems not to work in dried plasma is IFN-γ.

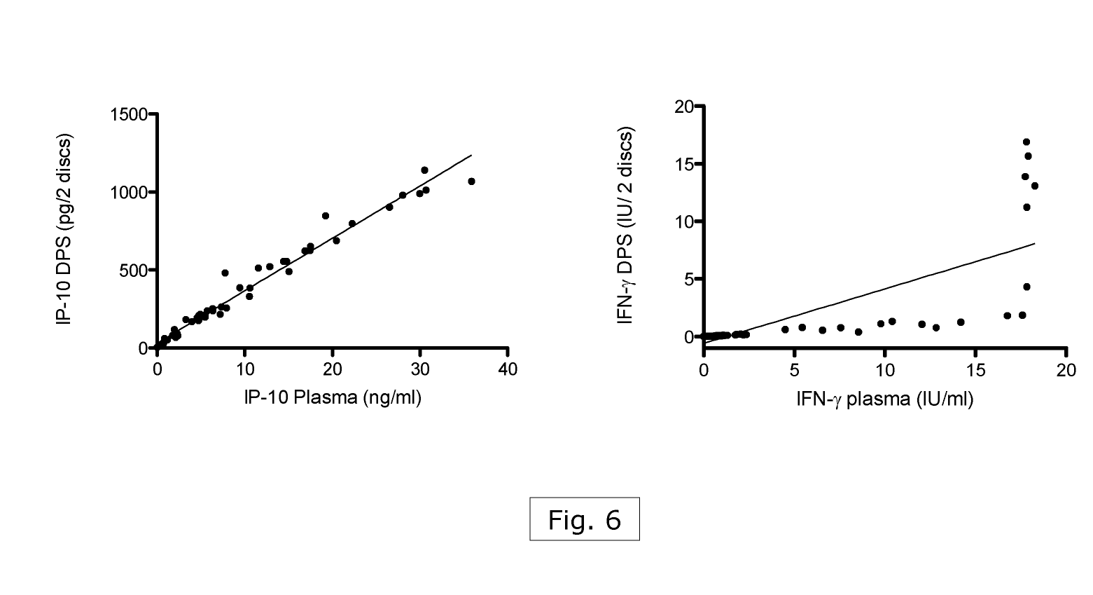

The poor performance of IFN-γ in DPS samples can further be appreciated in FIG. 6. In this graph we present the correlation between plasma and DPS. In the left panel 3 μL of plasma and 2 DPS discs are correlated for IP-10 and in right panel 50 μL of plasma and 2 DPS discs are correlated for IFN-γ. There was an excellent correlation between plasma and DPS IP-10 (r²=0.97) and a poor correlation for IFN-γ (r²=0.56). As is clear from the correlation plot for IFN-γ the plasma assay cannot fathom very high levels of plasma IFN-γ exceeding 17-18 IU/ml, but the DPS assay is able to present these extreme high levels of IFN-γ because the method represents a dilution compared to plasma.

### Example 6

Diagnosing Tuberculosis Test with ROC Data

Method: 3×1 ml of whole blood from 59 healthy controls never exposed to TB and whole blood from 60 patients with culture confirmed active tuberculosis was incubated at 37° C. for 18 hours in the Quantiferon in Tube system, 2×25 μL plasma was transferred to Whatman 903 paper and dried for 4 hours before storage at 2 weeks at 20° C. in sealed plastic bag with a desiccator as described in the General Method above. 2 discs were cut using the 6.0 mm standard hole punch and analyzed using the direct extraction and incubation method.

### Diagnostic Potential Independent of Concentration.

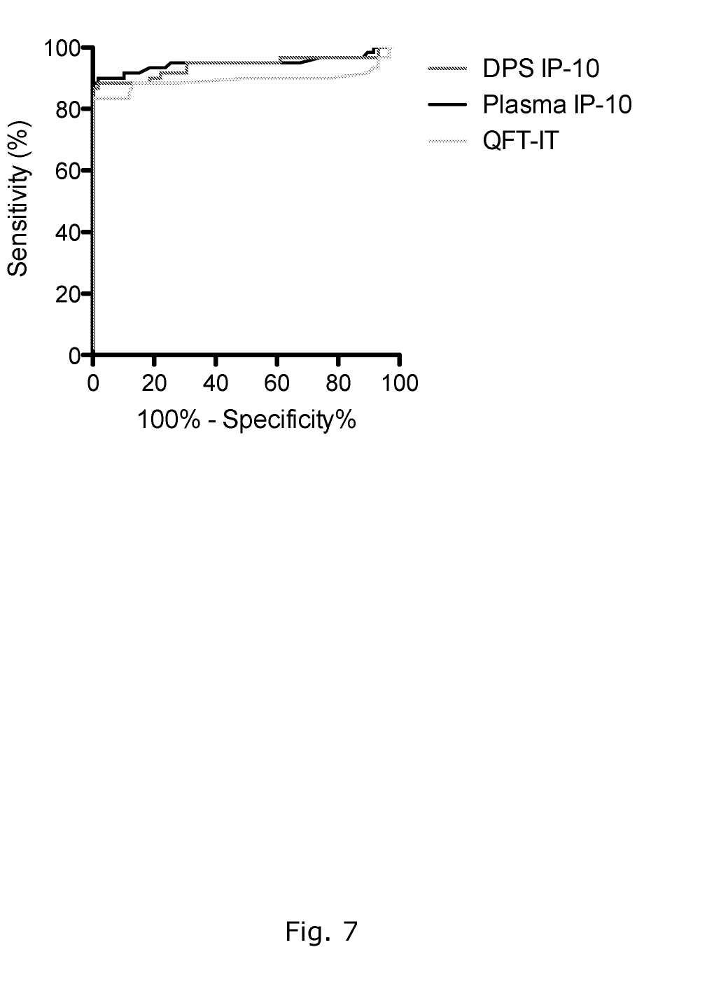

In FIG. 7 we present the ROC curves for the patients and controls comparing IP-10 detected in plasma and with the DPS method. The AUCs were comparable and slightly higher for IP-10: 0.95, 0.94 and 0.89, for IP-10 plasma, IP-10 DPS and QFT-IT, respectively.

### The Method as a Diagnostic Test.

The ROC analysis in FIG. 7 suggested the following cut off values for an assay with high specificity: IP-10 plasma 2.27 ng/ml (sensitivity 87%, specificity 100%) and IP-10 DPS 100 pg/2 discs (sensitivity 87%, specificity 100%) for comparison the optimal cut off for QFT-IT on these data was 0.21 IU/ml (sensitivity 83%, specificity 100%) Vis-à-vis the option in the QFT-IT test algorithm we defined a cut off for indeterminate test. We chose the cut off values for indeterminate test at the highest value that did not result in indeterminate responders among the controls: 1.44 ng/ml for IP-10 in plasma and 75 pg/2 discs for IP-10 in DPS. For analysis of IFN-γ results, the QFT-IT algorithm was used as recommended by the manufacturer. In table 5 we compare the diagnostic potential based on the algorithm suggested above and demonstrate comparable diagnostic accuracy between IP-10 plasma, IP-10 DPS and the Quantiferon test (QFT).

TABLE 5
diagnostic accuracy of the IP-10 DPS method compared to plasma
IP-10 and QFT.

| | | IP-10 plasma | IP-10 DPS | QFT-IT |
|---|---|---|---|---|
| TB patients | Positive | 61 (78) | 58 (74) | 58 (74) |
| | Negative | 11 (14) | 12 (15) | 16 (21) |
| | Indeterminate | 6 (8) | 8 (10) | 4 (5) |
| Controls | Positive | 2 (2) | 2 (2) | 0 (0) |
| | Negative | 95 (97) | 95 (97) | 98 (100) |
| | Indeterminate | 1 (1) | 1 (1) | 0 (0) |

### Example 7

IP-10 detection in dried blood spots (DBS) following CMV peptide stimulation. Method. 3×1 ml blood samples were taken from one healthy donor and stimulated with cytomegalovirus (CMV) peptides, phytohaemaglutinin (PHA) and saline (nil/neg control). Samples were incubated 24 hours at 37 C where after spots of 25 μL blood was dried on filter paper and stored at room temperature for 2 days.

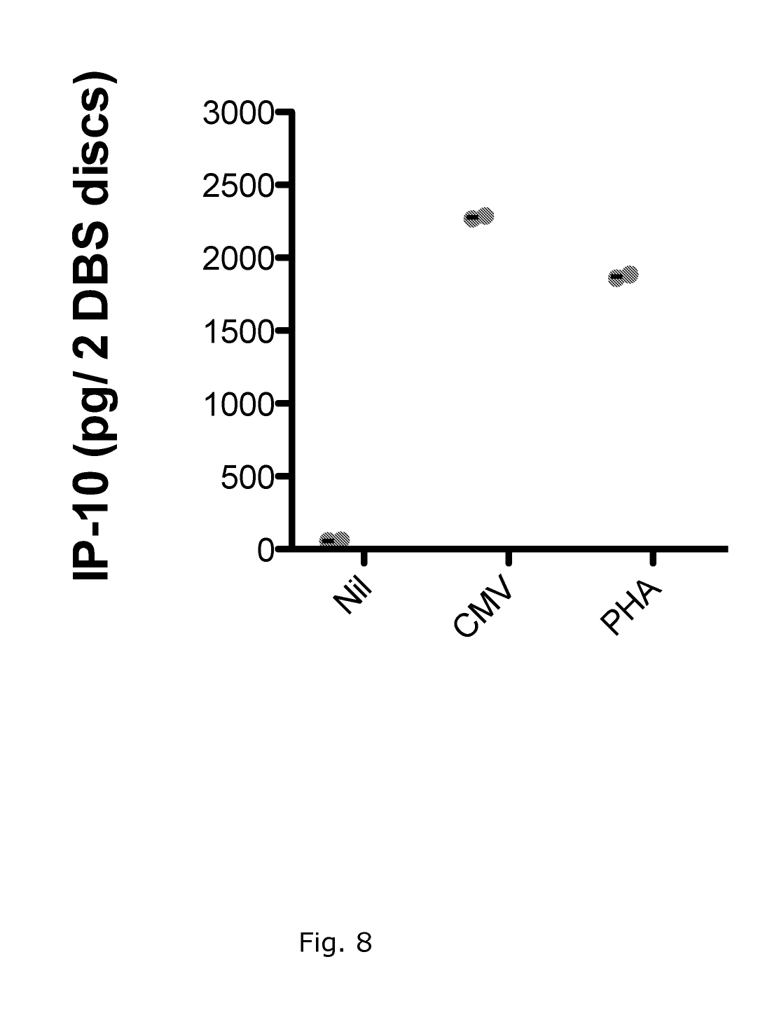

Results: in FIG. 8 the responses from two parallel experiments clearly demonstrating high responses to CMV peptides and PHA in the donor, and that these responses were clearly detectable in 2 DBS discs.

### Example 8

IP-10 detection in dried whole blood spots after TB10.4 and PHA stimulation. Method. Whole blood from 4 donors was stimulated with saline, TB10.4 peptides (15 mers overlapping) and PHA in 0.2 ml culture in a 1 ml syringe (COBAS, Roche, Switzerland). The syringe was prepared by adding the antigen or mitogen in stable from on a small filter paper disc. Following, the 200 μL blood sample was drawn from a heparized vaccutainer which had drawn 4 ml of blood. This allowed for instant easy mixing of peptides/mitogen and blood. IP-10 was detected in 2×5.5 mm DBS spots using the IP-10 ELISA as described above.

### Results.

In table 6 we appreciate that the 4 donors respond in high levels to TB10.4 and PHA, and that the responses are detectable with the DBS method also when stimulated in very low volumes of blood.

TABLE 6
Nil, PHA and TB10.4 responses (in pg/2 discs) from 4 donors detected
using the DBS method.

| | Donor 1 | Donor 2 | Donor 3 | Donor 4 |
|---|---|---|---|---|
| Nil | 26 | 355 | 15 | 21 |
| TB10.4 | 40 | 413 | 49 | 121 |
| PHA | 91 | 381 | 122 | 169 |

### Example 9

### A Comparison of the Signal Intensity in DPS, PBS and Plasma

For this example we took 4 donors and stimulated whole blood in the Quantiferon nil, CMV and PHA tubes and determined IP-10 in 3.03 μL plasma (×33 diluted) and in 2 DPS and 2 DBS spots.

### Results:

In table 7 we appreciate the OD readings, these are well within the working range in of the assay especially for responses in the range of the cut off.

TABLE 7
Signal intensity in OD generated from the three different types of
samples in the 4 donors.

| | | Donor 1 | Donor 2 | Donor 3 | Donor 4 |
|---|---|---|---|---|---|
| Plasma (pg/ml) | Nil | 0.021 | 0.121 | 0.019 | 0.007 |
| | CMV | 1.053 | 2.908 | 2.912 | 0.010 |
| | PHA | 1.067 | 1.741 | 0.720 | 1.813 |
| DBS (pg/2 discs) | Nil | 0.061 | 0.301 | 0.072 | 0.067 |
| | CMV | 1.502 | 3.362 | 3.363 | 0.062 |
| | PHA | 1.624 | 2.198 | 1.120 | 2.456 |
| DPS (pg/2 discs) | Nil | 0.049 | 0.256 | 0.019 | 0.006 |
| | CMV | 1.607 | 3.098 | 2.964 | 0.011 |
| | PHA | 1.558 | 2.445 | 0.732 | 1.835 |

In table 8 we appreciate the derived concentrations after correcting for dilution of the plasma sample. It is cardinal to appreciate that there is a huge difference in magnitude of response from plasma to the filter paper (24.8 and 24.4 fold higher responses in the plasma samples compared to DBS and DPS, (only including samples PHA and CMV)), but that the difference between DPS and DBS is very small.

TABLE 8
Concentration of IP-10 generated from the three different types of
samples in the 4 donors.

| | | Donor 1 | Donor 2 | Donor 3 | Donor 4 |
|---|---|---|---|---|---|
| Plasma (pg/ml) | Nil | 243 | 1434 | 225 | 77 |
| | CMV | 12476 | 34471 | 34513 | 119 |
| | PHA | 12642 | 20638 | 8535 | 21485 |
| DBS (pg/2 discs) | Nil | 22 | 108 | 26 | 24 |
| | CMV | 540 | 1208 | 1208 | 22 |
| | PHA | 583 | 790 | 402 | 882 |
| DPS (pg/2 discs) | Nil | 18 | 92 | 18 | 8 |
| | CMV | 577 | 1113 | 1188 | 11 |
| | PHA | 560 | 878 | 425 | 932 |

In other words when determining the concentration of a biomarker in 2 DBS or 2 DPS samples these correspond to plasma diluted ×24.8 and 24.4 i.e approx. 4 μL of plasma per 100 μL ELISA well. As one filter paper disc with a radius of 5.5 mm has the area of 23.8 mm² (and two disc have the area of 47.6 mm²), then 1 μL plasma generates a signal corresponding to 11.9 mm² of DPS or DBS if analyzed in a 100 μL ELISA well (47.6 mm²/4 μL).

### Example 10

Comparison of the Recovery in a Representative Set of LPS Stimulated Samples, Which were within Range of the Assay in Both Plasma and DPS Samples.

Whole blood from three representative donors was stimulated with LPS and serially diluted in un-stimulated plasma to generate samples with variable IP-10 and IFN-γ content and constant volume of plasma. Plasma samples were measured at ×33 dilution (corresponding to 3.03 μL plasma in the 100 μL assay well) plasma measurements were not corrected for dilution, this to facilitate comparison with DPS measurements. Data are presented in table 9. Interpretation: The IP-10 concentration in 2 DPS discs was 1.37 (s.d. 0.36) fold higher than the 3.03 μL plasma sample, corresponding to a mean plasma volume of (1.372×3.034) 4.24 (+/−1.14) recovered from each set of 2 discs.

TABLE 9
head to head comparison of IP-10

| | DPS (2 disc) | Plasma (pg/ml) | DPS/Plasma |
|---|---|---|---|
| Donor 1 | 206 | 225 | 0.9 |
| | 119 | 111 | 1.1 |
| | 72 | 61 | 1.2 |
| | 38 | 31 | 1.2 |
| | 22 | 15 | 1.4 |
| Donor 2 | 144 | 135 | 1.1 |
| | 79 | 68 | 1.2 |
| | 44 | 44 | 1.0 |
| | 27 | 20 | 1.4 |
| | 18 | 9 | 2.1 |
| | 9 | 5 | 1.7 |
| Donor 3 | 257 | 194 | 1.3 |
| | 136 | 96 | 1.4 |
| | 74 | 48 | 1.5 |
| | 40 | 25 | 1.6 |
| | 26 | 12 | 2.2 |
| | 7 | 6 | 1.1 |
| | | Average | 1.4 |
| | | s.d. | 0.4 |

For comparison we performed the same analysis on IFN-γ (table 10). All but 3 DPS samples had IFN-γ levels below the LOQ of the Quantiferon ELISA assay (0.2 IU/ml), based on these 3 samples (marked in bold) we calculated a mean recovery of 0.071 fold corresponding to a plasma volume of (0.071×504) 3.64 (+/−0.6 μL) for IFN-γ (p=0.11 compared to the volume calculated using IP-10). It is evident from this table that whenever the DPS readings get below 0.2 IU/ml then there is a dramatic increase in DPS/plasma ratio simply illustrating that this assay is not suitable for reliable detection of concentrations below this point.

| | Plasma 50 uL | DPS (IU/2 spots) | DPS/plasma |
|---|---|---|---|
| Donor 1 | 5.31 | 0.34 | 0.064 |
| | 2.56 | 0.19 | 0.074 |
| | 1.17 | 0.11 | 0.094 |
| | 0.59 | 0.07 | 0.119 |
| | 0.29 | 0.06 | 0.207 |
| | 0.03 | 0.04 | 1.333 |
| Donor 2 | 2.02 | 0.11 | 0.054 |
| | 0.91 | 0.08 | 0.088 |
| | 0.47 | 0.05 | 0.106 |
| | 0.24 | 0.05 | 0.208 |
| | 0.15 | 0.04 | 0.267 |
| | 0.04 | 0.04 | 1.000 |
| Donor 3 | 9.7 | 0.76 | 0.078 |
| | 5.27 | 0.34 | 0.065 |
| | 2.58 | 0.22 | 0.085 |
| | 1.19 | 0.13 | 0.109 |
| | 0.62 | 0.08 | 0.129 |
| | 0.03 | 0.04 | 1.333 |

| | |
|---|---|
| Average | 0.301 |
| S.d. | 0.433 |
| Selected 3 average | 0.071 |
| s.d. | 0.012 |

In conclusion, the recovery of IP-10 and IFN-γ from 2 DPS discs of 5.5 mm using this method was the same. The recovery corresponds to a plasma volume of 4.2 μL per 100 μL assay well (=24 fold dilution of the sample). The vast majority of IFN-γ signals recovered from DPS samples were below the LOQ of the IFN-γ ELISA illustrating the problems of applying the DPS/DBS method for IFN-γ.

1. A method for measuring an antigen specific cell-mediated immune response comprising the steps of:
a) incubating a sample comprising T-cells obtained from a mammal with at least one antigen;
b) applying a fraction of the sample on filter paper;
c) determining the level of IP-10 in a fraction of said filter paper; and
d) comparing said determined level of IP-10 in said filter paper with a reference level, thereby determining whether the mammal has previously encountered the first antigen generating immunological reactivity to the first antigen or previously encountered other antigens generating immunological cross reactivity to the first antigen.

2-22. (canceled)

23. The method according to claim 1, wherein the sample is divided into at least 2 fractions and:
a) incubating the first fraction of the sample with the antigen to generate a response sample;
b) incubating the second fraction of the sample with an inactive solution to generate a nil sample;
c) applying a fraction of the response sample on filter paper;
d) applying a fraction of the nil sample on filter paper;
e) determining the antigen dependent IP-10 response by subtracting the IP-10 level determined in the filter paper from the nil sample from the IP-10 level determined in the filter paper from the response sample; and
f) comparing the antigen dependent biomarker response or a value derived thereof with the reference level or a value derived thereof, thereby determining whether mammal has previously encountered the first antigen and thus generate immunological reactivity to the first antigen or previously encountered other antigens generating immunological cross reactivity to the antigen.

24. The method according to claim 23, further comprising dividing the sample into 3 fractions and incubating the third fraction of the sample with a T cell activator to generate a positive control sample followed by the application of a fraction of the positive control sample on filter paper.

25. The method according to claim 1, wherein the antigen is specific for a microorganism.

26. The method according to claim 1, wherein the antigen is specific for a bacterium.

27. The method according to claim 1, wherein the antigen is specific for a virus.

28. The method according to claim 26, wherein the antigen is specific for Mycobacteria.

29. The method according to claim 26, wherein the antigen is specific for Mycobacterium tuberculosis.

30. The method according to claim 26, wherein the antigen is selected from the group consisting of antigens comprising RD-1 antigens, RD-11 antigens, ESAT-6, CFP-10, TB7.7, TBCELLSAT, and Rv3615c.

31. The method according to claim 1, wherein the antigen is a peptide and/or a protein and/or a panel of at least 2 peptides.

32. The method according to claim 1, wherein the IP-10 level is determined in a fraction or several fractions of the filter paper with a known size.

33. The method according to claim 1, wherein the IP-10 level is determined by elution of the biomarker from the filter paper in a buffer.

34. The method according to claim 1, wherein the IP-10 level is determined by elution of the biomarker and detecting the biomarker in one step.

35. The method according to claim 34, wherein the elution and detection steps are done using pairs of monoclonal antibodies binding the biomarker so as to create a sandwich.

36. The method according to claim 35, wherein the sandwich is coupled to an enzyme, a molecule capable of generating chemiluminescence or a means for signal amplification.

37. The method according to claim 35, wherein the sandwich binds a solid phase.

38. The method according to claim 1, wherein the antigen is a pool of peptides specific for Mycobacterium tuberculosis, and the IP-10 level is eluted and determined using ELISA.

39. The method according to claim 1, wherein the sample is whole blood, plasma or another blood derived component.

40. The method according to claim 1, wherein the mammal is a human.

41. The method according to claim 1, wherein the incubation is done at a temperature from 37° C. to 39.5° C.

42. The method according to claim 41, wherein the incubation is done at 39° C.

43. The method according to claim 1, wherein the level of IP-10 is detected as messenger RNA.
